(19) United States
(12) Patent Application Publication
(10) Pub. No.: US 2026/0294755 A1
(43) Pub. Date: Oct. 1, 2026

(54) COMPOSITION FOR DELIVERING PARTICLES TO FOLLICLES

(71) Applicant: The Gillette Company LLC, Boston, MA (US)

(72) Inventors: Ilaria Ambrogio, London (GB); David Peter Andrew Muir, Windsor (GB); Daniel Edward Dunham, Wiltshire (GB); Martin Stephen Williamson, Bracknell (GB); Ignacio De Miguel Clave, Barcelona (ES)

(21) Appl. No.: 19/636,033

(22) Filed: Apr. 1, 2026

Related U.S. Application Data
(60) Provisional application No. 63/781,777, filed on Apr. 1, 2025.

(51) Int. Cl. A61K 8/25; A61K 8/02; A61K 8/04; A61Q 7/00

(52) U.S. Cl. CPC A61K 8/25; A61K 8/0245; A61K 8/044; A61Q 7/00

(57) ABSTRACT

This application relates to particles and other light absorbing materials optimal for insertion into pores for light based hair removal. The composition has a host particle coated with metal nanoparticles.

### FIELD

The present invention relates to a nanoparticle adsorbed on a host microparticle in a composition intended to deliver particles to hair follicles in the dermis. The composition may be used for delivering actives or for delivery for the purpose of treatments such as, for example, photoepilation.

### BACKGROUND

There are many lotions and treatments that rely on delivering actives to follicles in the dermis. Further, cosmetic treatments such as photoepilation may benefit from the delivery of particles to the hair follicle. Photoepilation (also known as light hair removal) is a non-surgical cosmetic procedure that uses intense light to remove unwanted hairs and slow down their regeneration. Light hair removal (including laser and IPL-Intense Pulsed Light) is the fastest growing non-surgical esthetic application.

Current approaches for photoepilation rely on the chromophore present in the hair follicle and on the natural color (absorption) contrast between the hair and the skin to damage the hair follicle by photo heating. They consequently may fail for lightly/non pigmented hairs (blond white, grey and red) and, even in the ideal configuration of dark hairs on light skin.

A method for enhanced photoepilation was described in the patent application WO2013079105 in which plasmonic gold nanocomplexes are used to artificially enhance the absorption contrast between the hair and the skin. In this method cationic gold nanocomplexes able to accumulate at the hair cuticle and root levels are used. Those nanocomplexes present a strong surface plasmon resonance (SPR) in the near infrared and thus can be heated up very efficiently at wavelengths out of the melanin absorption spectrum. However, in practice, only a finite number of nanocomplexes will reach the hair cuticle/root, thus limiting the actual depilation efficiency, because the quantity of heat generated at the hair cuticle/root is directly proportional to the quantity of delivered nanocomplexes. In addition, nanocomplexes belong to nanotechnologies and from a commercial/societal viewpoint, they may raise some issues related to tiny size like potential toxicity or clearance.

As such, there is a need to increase the efficiency and specificity of delivery for nanocomplexes to the hair follicle in the dermis.

### SUMMARY

This application relates a composition intended to deliver the particle to hair follicles in the dermis, the composition comprising a host particle exemplified herein as silica particles and metal particles, the composition exhibits a greater than 70% attachment of the metal particles to the silica particles. A further embodiment of the invention relates to a composition intended to deliver the particle to follicles in the dermis, the composition comprising silica particles and metallic nanorod particles at an attachment level of greater than 70%.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIG. 1 shows images of Anionic Silica at a magnification of 50×.

FIG. 2 shows Cationic Gold Nanoparticles at a magnification of 80×.

FIG. 3 shows Anionic Silica coated with Gold Nanoparticles at a magnification of 80×.

FIG. 4 shows a higher resolution portion of FIG. 3.

FIG. 5 shows the Two Photon Luminescence (TPL) image of a hair root incubated with gold-chitosan nanocomplexes penetration of a Nanoparticle-Gel Network.

FIG. 6 shows the Two Photon Luminescence (TPL) image of a hair root incubated with gold-chitosan nanocomplexes penetration of a Nanoparticle Suspension.

FIG. 7 shows the Two Photon Luminescence (TPL) image of a hair root incubated with gold-chitosan nanocomplexes penetration of a Microparticle Suspension.

FIG. 8 is a chart for Depth Achieved from Different Massage Types.

FIG. 9 is a chart for Follicle Delivery from Different Massage Types.

### DETAILED DESCRIPTION

As mentioned above, an aspect of the present invention relates to a composition intended to deliver the particle to hair follicles in the dermis. The composition comprises a nanoparticle adsorbed on a host microparticle. The composition may be used in photoepilation. The composition comprises a nanocomplex which comprise a nanoparticle of a material selected from the group consisting of metals, semiconductors and their mixtures, supporting a Localized Surface Plasmon Resonance, which is coated by forming at least a bond, coordinate or covalent, with at least a polycationic polymer or a heterobifunctional compound which comprises at least a thiol group on one end of the molecule and at least a positively charged functional group on the other end; adsorbed on the surface of a host particle with a diameter comprised between 100 nm and 2000 nm; the nanocomplex have a positive Z potential higher than 10 mV.

The invention relates to a composition that comprises the nanocomplex of the invention which are able to penetrate, upon application on the skin surface, into the hair follicle and reach the target cells.

Prior to application of the method, skin and hair follicles can be pretreated to increase the delivery of the nanocomplex of the invention to a target region. In some embodiments, hair shafts are cut or removed via shaving, waxing or other techniques to remove hair. In some embodiments pre-treatment with surface exfoliation including mechanical exfoliation and chemical exfoliation removes plugs from the orifice of follicles to increase the targeting of nanocomplex to target region within the hair follicle. In addition, a pretreatment with hot water or steam can be useful to open the hair follicle.

Thus, another aspect refers to a photoepilation method which comprises the steps of (i) applying to the skin surface the composition for topical application as defined above, and (ii) applying a radiation to said surface of the skin.

The radiation used in step (ii) can be from any light source whose emission presents a spectral overlap with the LSP resonance of the nanocomplex comprised in said composition.

Examples of light sources are those conventionally used in photoepilation methods, such as Intense Pulsed Light (IPL) or continuous wave and pulsed laser light. Other source of light as Light Emitting devices (LED) is also considered.

Finally, another aspect of the present invention relates to the use of the nanocomplex of the invention in a photoepilation method.

The term “host particle” as used herein refers to particles which are capable of bonding to metals. The host particles may utilize electrostatic bonding, covalent bonding, hydrogen bonding or any other known form of bonding in the art. In an embodiment, the host particles are preferably spherical in form or essentially spherical in form. In an embodiment, the host particles may be selected from a ceramic. The host particles may comprise of silica, zirconia, alumina, polystyrene, polystyrene sulfinate, and combinations thereof. In an embodiment, the host particle exhibit and opposing charge to the metal particle being attached to the host particle. In an embodiment, the host particle is made of silica, modified silica, cellulose, modified cellulose as for example carboxymethyl cellulose or particles made of polyanionic polymers such as polystyrene sulfonate or polyacrylates. Preferred the host particle is made of silica or activated silica microparticles or surface modified silica microparticles (either with carboxylate, sulfonate, amine groups, epoxy groups or other organic groups). In a particular embodiment the host particle is made of activated silica and in a second particular embodiment the host particle is made of silica modified with amine groups.

In a more preferred embodiment, the diameter of the host particle is comprised between and inclusive of 200 nm and 2000 nm, such as, for example 250 nm, 300 nm, 350 nm, 400 nm, 450 nm, 500 nm, 600 nm, 650 nm, 700 nm, 750 nm, 800 nm, 850 nm, 900 nm, 950 nm, 1000 nm, 1050 nm, 1100nm, 1150 nm, 1200 nm, 1250 nm, 1300 nm, 1350 nm, 1400 nm, 1450 nm, 1500 nm, 1550 nm, 1600 nm, 1650 nm, 1700 nm, 1750 nm, 1800 nm, 1950 nm, and 2000 nm.

### Metal Particle

The metal particle(s) may be gold or silver particles or combinations thereof, such as gold or silver nanorod particles. In a particular embodiment the metal nanoparticle is a gold nanoparticle, such as, for example, a gold nanorod. Recent numerical studies enabled to quantify the influence of the geometry of gold NPs on its heating ability. It was demonstrated that the optical field intensity within the metal is the main parameter to be maximized in order to increase the temperature of the NP. This is the reason why, for a given illumination, elongated gold NPs (known as nanorod (NR)) generate more heat than gold nanospheres of the same volume.

Considering all the above-mentioned parameters, one can design gold NRs to act as efficient punctual heat-sources to be remotely controllable by light. Importantly, the temperature profile associated to a punctual heat source decays in 1/r, where r is the distance from the source. Therefore, according to a preferred embodiment, the micro complex of the invention comprises a gold nanorod.

The silver nanorod may be between and inclusive of 10 nm in diameter to 40 nm in diameter and between and inclusive of 50 nm in length to 90 nm in length. The silver nanorod particles may have a length to thickness ratio of greater than or equal to 2.5.

The Gold nanorod may be between and inclusive of 10 nm in diameter to 40 nm in diameter and between and inclusive of 50 nm in length to 90 nm in length. The gold nanorod particles may have a length to thickness ratio of greater than or equal to 2.5.

It has been surprisingly found, as discussed below, that by controlling pH, the ratio of metal particles, here exemplified by gold nanorods, to silica inclusive of the ratio between and size of the particles, and suspension hostility through additives, one can dial in penetration depth of the silica particles coated in gold nanorods.

According to another embodiment of the invention said chemical compound specifically binds through receptor-mediated processes, to a molecule target present on the basal cells of the hair. Examples of said compounds in this case are antibodies, which can be obtained and thereafter chemically modified to be grafted to the NP surface according to well-known methods.

According to a particular embodiment the composition is an aqueous suspension that is cosmetically or pharmaceutically acceptable comprising of a pH between and inclusive of 6.5 and 7.3 such as, for example, 6.6, 6.7, 6.8, 6.9, 7, 7.1 and 7.2. The composition may however be also in form of a suspension, a gel, milk, a cream, an ointment, a lotion, a foam, a soft paste, or any other cosmetically acceptable form provided that the composition pH remains between 6 and 8.

The micro complexes are present in a cosmetically efficient amount. Under cosmetically efficient amount is to be understood the minimum amount to particle deposition to the dermis. The composition may further contain other cosmetically or pharmaceutically acceptable ingredients such as solvent or cosmetic additives or vehicles. In an embodiment, the concentration in water of the active ingredients may be 300 ppm for Au and 750 ppm for Si. In an embodiment, the composition may further comprise an additive such as LAE. LAE is a food preservative, broad spectrum antimicrobial compound, included to reduce yeast and mould susceptibility. LAE may be at a concentration of between 0.05% LAE and 0.5% LAE such as, for example, 0.06% LAE, 0.07% LAE, 0.08% LAE, 0.09% LAE, 0.1% LAE, 0.2% LAE, 0.3% LAE, 0.4% LAE, or 0.5% LAE. The composition may further comprise Benzyl Alcohol (BA) which is an antibacterial with lower efficacy for yeast and mould. The Benzyl Alcohol may be at a concentration of between 0.3% to 1%, such as, for example, 0.5% BA, 0.55% BA, 0.6% BA, 0.75% BA, 0.75% BA, or 1% BA. It has been found that additives impact the hostility of the suspension. In relation to hostility, without being bound by theory, it is believed that too much hostility leads to detachment of the gold nanorods from the silica particles while not sufficient hostility leads to poor attachment. Additives may further comprise of organic acids/salts such as Benzoic acid, Salicylic Acid, or Dehydroacetic Acid; Isothiazolinones such as MIT; Parabens such as Methyl, Ethyl, propyl parabens; or other preservatives such as, 1,2-Hexane diol, phenoxyethanol or any combination thereof.

### Ratio

It has been found that a ratio of host particle to metal nanoparticle of between and inclusive of 1.5:1 to 6.5:1 in relation to the concentration (ppm: ppm), such as for example 2:1, 3:1, 4:1, 5:1, and or 6:1 in relation to the concentration (ppm: ppm) leads to surprising results in terms of particle deposition as shown in the figures.

The formation of the particles may be done by the following steps:
Adding Ultra-pure H2O to an Si solution, then treating the solution with a pH modifier to decrease the pH of the solution to create a Neutral to Basic pH solution. Au solution is treated with a pH modifier to increase the pH of the solution to create an Acidic pH solution; preferably with a pH less than 6.7. Combining the Si solution and Au solution. Adding a buffering agent to the solution, to create a pH Neutral to Basic Solution. Optionally, affecting particle distribution using any of sonication, mixing, introduction of energy into the system (kinetic or thermal), milling, or any other means known in the art. Preferably, affecting particle distribution is done through sonication. Optionally, allowing the Neutral to Basic pH solution to rest for a fixed period of time, followed by additional agitation. And, adding preservatives. Preferably, LAE and Benzyl Alcohol.

### Example 1

The particles may be formed by the following procedure wherein the SiMP and AuNP are buffered separately prior to be combined. In an embodiment, the AuNP may be slightly acidic such as, e.g., with a pH greater than 6 and below or equal to 7. The SiNP may be slightly basic such as, e.g., with a pH greater than 7. The SiMP and AuNP are combined followed by an additional buffering. The combination is then sonicated for 2 minutes and left to mature for 24-48 hours (room temperature, static). After maturing, preservatives may be added such as LAE & Benzyl Alcohol. In the example below, the LAE concentration is 0.1% and the Benzyl Alcohol concentration is 0.5%.

The suspension may have a viscosity (cP) equal to or substantially the same as water. The suspension may have a value for viscosity (cP) as the particles should give some measurable resistance of 16000 cp to 28000 cp.

Below is a table of viscosities for samples:

| | Zero-rate | |
|---|---|---|
| Sample | viscosity (Pas) | cP |
| 300 ppm MCB011 | 0.041175 | 41.175 |
| 300 ppm MCB010 0.1% LAE 0.5% BA | 0.040564 | 40.564 |
| 0.2% Actigum | 27.7686 | 27768.6 |
| 0.3% Actigum | 16.2588 | 16258.8 |

The particles may be delivered to the hair follicles in the dermis by rubbing the composition on the skin and allowing the particles to penetrate. The delivery does not necessitate mechanical methods or agitation such as massaging, or the use of acoustic, ultrasound, alternating suction and pressure, or jets.

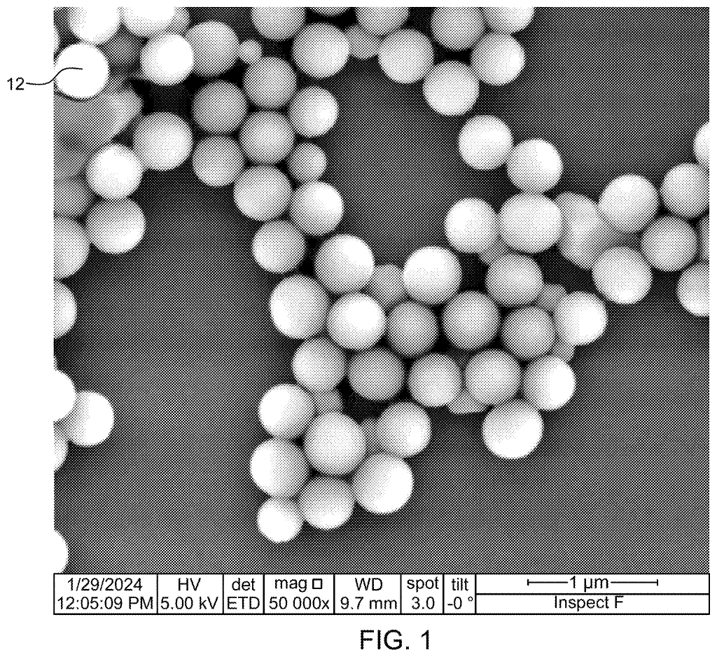

FIG. 1 shows a Scanning Electron Microscope (SEM) image of the anionic silica particles. As seen in FIG. 1, the particles 12 are spherical and do not have any coating on the outer shell.

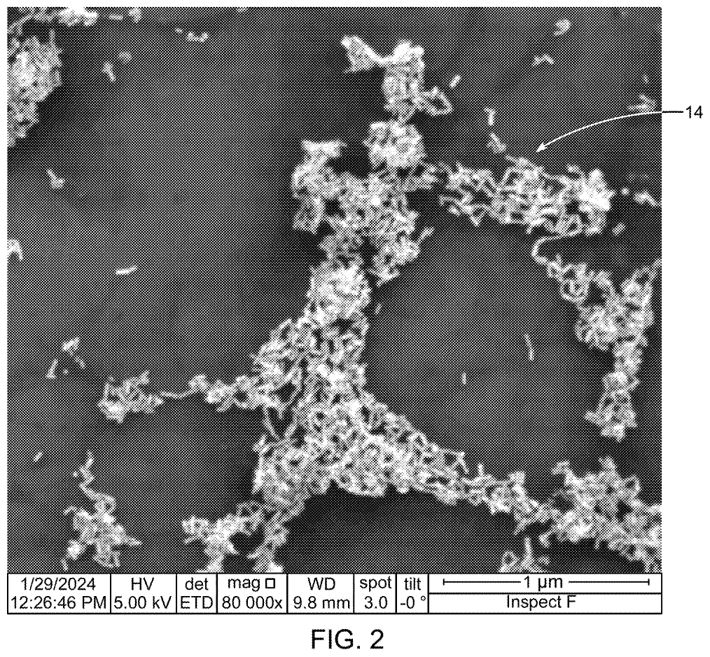

FIG. 2 shows a SEM of gold nanoparticles 14. The gold particles are in the form of gold nanorods.

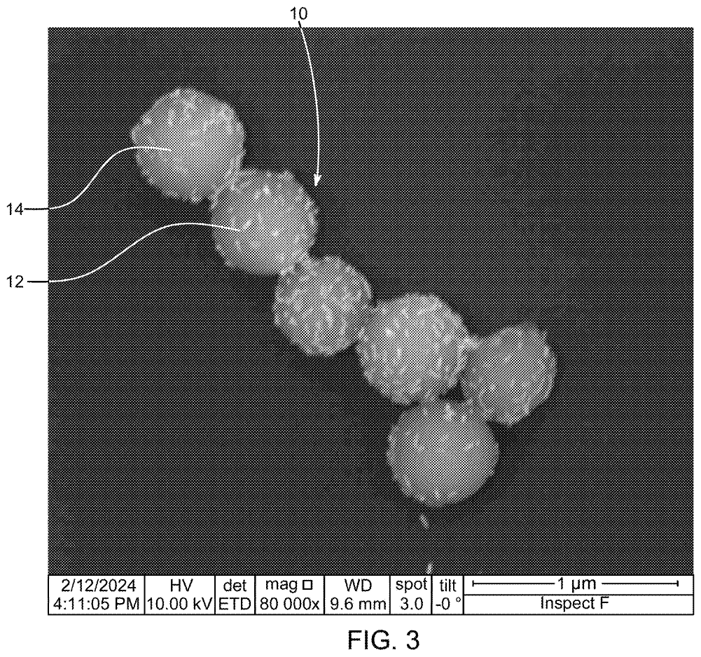

FIG. 3 shows an SEM of the coated particles 10 being the anionic silica particles 12 of FIG. 1 coated with the gold nanorod particles 14 of FIG. 2.

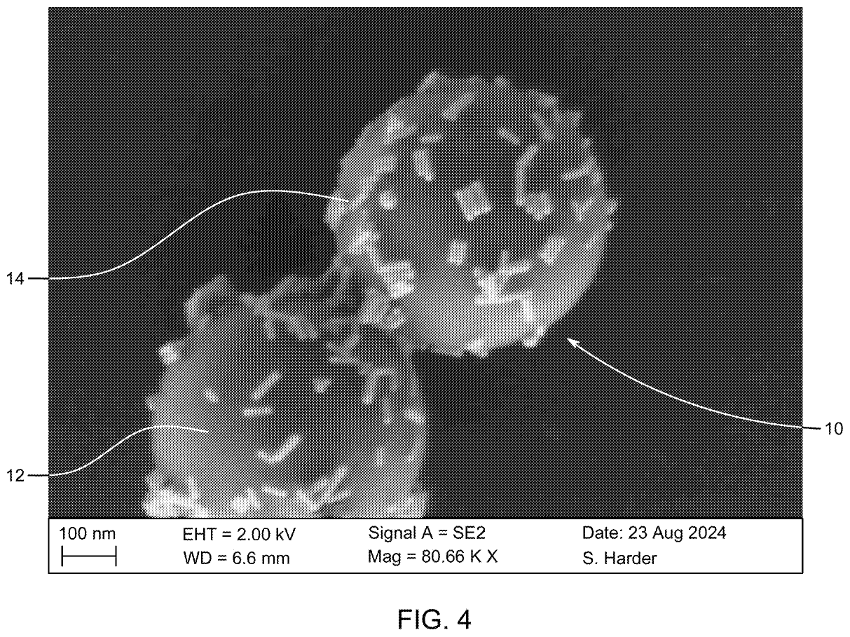

FIG. 4 is an SEM of silica spherical particles coated with gold nanorods.

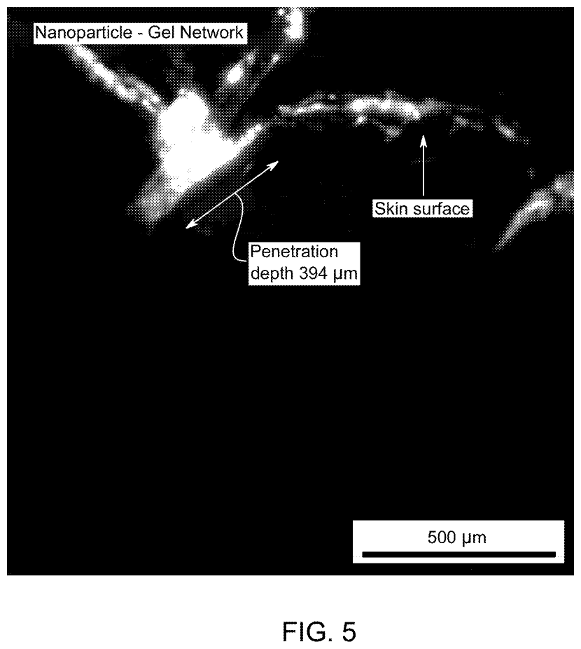

FIG. 5 shows the Two Photon Luminescence (TPL) image of a hair root incubated with gold-chitosan nanocomplexes penetration of a Nanoparticle-Gel Network. As shown in FIG. 5, by using a nanoparticle gel network comprising of the appropriate ratios at the proper pH, one can achieve deep penetration depth without the use of additional mechanical tools.

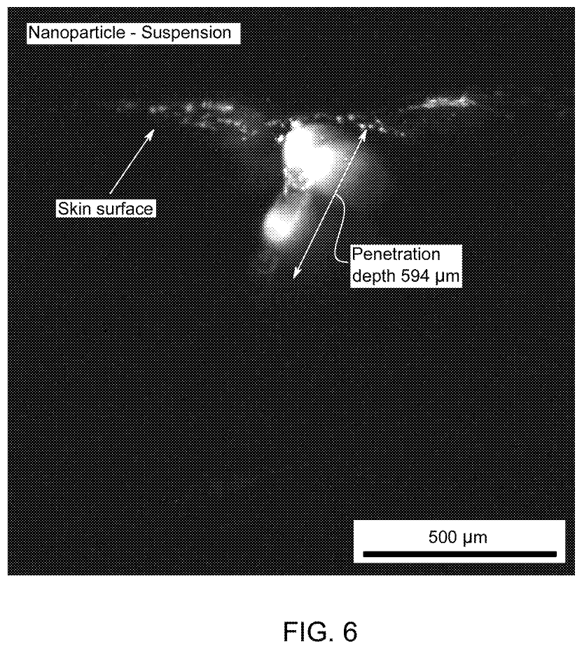

FIG. 6 shows the Two Photon Luminescence (TPL) image of a hair root incubated with gold-chitosan nanocomplexes penetration of a Nanoparticle Suspension. As shown in FIG. 6, by using a nanoparticle suspension comprising of the appropriate ratios at the proper pH, one can achieve deep penetration depth without the use of additional mechanical tools. The suspension penetrated the skin by over 500 micrometers.

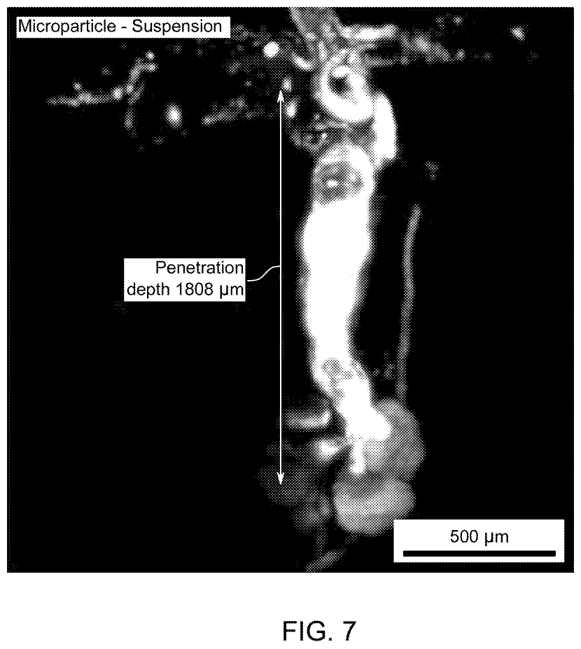

FIG. 7 shows the Two Photon Luminescence (TPL) image of a hair root incubated with gold-chitosan nanocomplexes penetration of a Microparticle Suspension. As shown in FIG. 7, the microparticle suspension penetrated the dermis by over 1800 micrometers.

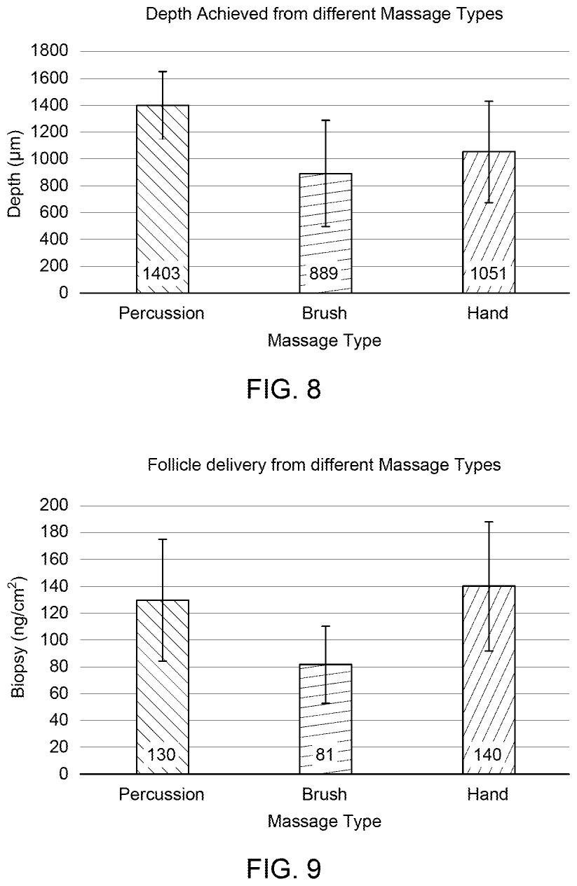

FIG. 8 is a chart for Depth Achieved from Different Massage Types. As shown in FIG. 8, by utilizing an embodiment of the invention, when deposited on the skin, the composition allows for deep penetration without the use of additional mechanical forces.

Further, as shown in FIG. 9, one can achieve equivalent if not greater delivery to the follicle with the composition described herein. When viewing FIGS. 8 and 9, it is evident that, while mechanical force such as percussion may allow one greater depth, composition of the lotion and the correct pH is more significant when it comes to delivery to the follicle once the composition has penetrated the dermis.

The composition may be in the form of a suspension, a gel, milk, a cream, an ointment, a lotion, a foam, a soft paste, or any other cosmetically acceptable form.

### Attachment Determination Method

Samples containing nanoparticles are first mixed thoroughly to eliminate visible precipitates. Equal volumes of reference nanocomplexes and test samples (microcomplex) are loaded into Vivaspin centrifuge tubes. 0.1% LAE is added to the microcomplex samples. All samples are then centrifuged at 20° C., 4500 rpm (or 1500 g) for 25 minutes.

After centrifugation, the filtrates are collected, and a mild surfactant (0.5% LAE solution) is added to each tube, followed by vortexing to ensure uniform mixing. The extent of nanoparticle attachment is quantified by comparing the UV spectral area of nanoparticles filtered from microcomplex suspensions to that from nanoparticle-only suspensions. The attachment percentage is calculated as follows:
Attachment %=1−(Area from microcomplex filtrate/Area from nanoparticle filtrate)
To validate the experiment (i.e. confirm the filtration was successful), at least 95% of the nanoparticle in the reference nanoparticle only solutions passes through the filter/is recovered in the filtrate. A successful microparticle is one that has an attachment of >90%.
Dilutions are prepared consistently in 0.5% LAE water before measurement to standardize UV absorbance readings.

### Filters Used

Vivaspin 500 Centrifugal concentrators, with 500 μl volume, and a filter pore size of 0.2 um.

### Acronyms:

TEM=Transmission electron microscopy
ICFO=Institute of Photonic Sciences, Barcelona PPM=parts per million
LAE=Ethyl lauroyl arginate
SiMP=Silica Microparticle, product of Gold Nanoparticle+silica
AuNP=Gold Nanoparticle
cP=Centipoise
UV-VIS: Ultraviolet—Visible spectroscopy
Vivaspin: Specialised Type of centrifugation used to prep the samples
Turbiscan: type of dynamic light scattering technique used to assess stability of colloidal systems MST: Microbial susceptibility technique.

### Examples/Combinations

A. A composition intended to deliver the particle to follicles in the dermis, the composition comprising host particles and metal particles, wherein the composition exhibits a greater than 70% attachment of the metal particles to the host particles.
B. The composition of paragraph A, wherein the pH of the composition is between 6 and 8.
C. The composition of any of paragraphs A to B, wherein the composition further comprises a preservative.
D. The composition of paragraph C, wherein the preservative is selected from the group consisting of LAE, Benzyl Alcohol, MIT, Methyl, Ethyl, propyl parabens, Dehydroacetic Acid and 1,2-Hexane diol, phenoxyethanol and combinations thereof.
E. The composition of paragraph E, wherein the composition further comprises between 0.01% LAE and 1% LAE.
F. The composition of any of paragraphs D to E, wherein the composition comprises between 0.3% Benzyl Alcohol and 1% Benzyl Alcohol.
G. The composition of any of paragraphs A to F, wherein the host particles are silica particles, wherein the silica particles are between 300 nm to 1000 nm in diameter.
H. The composition of any of paragraphs A to G, wherein the ratio of host particle to metal nanoparticle of between and inclusive of 1.5:1 to 6.5:1.
I. The composition of any of paragraphs A to H, wherein the metal particles comprise gold nanorods, silver nanorods, or combinations thereof.
J. The composition of any of paragraphs A to I, wherein the metal particles are one or more gold nanorods, each of the gold nanorods having a peak max absorption of between 700 nm to 1100 nm.
K. The composition of any of paragraphs A to J, wherein the composition is in the form of a suspension, a gel, milk, a cream, an ointment, a lotion, a foam, a soft paste, or any other cosmetically acceptable form.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Combinations of components are contemplated to include homogeneous and/or heterogeneous mixtures, as would be understood by a person of ordinary skill in the art in view of the foregoing disclosure.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to anything disclosed or claimed herein or that it alone, or in any combination with any other reference or references, teaches, suggests or discloses anything disclosed or claimed herein. Further, to the extent that any meaning or definition of a term in this document conflicts with any meaning or definition of the same term in a document incorporated by reference, the meaning or definition assigned to that term in this document shall govern.

While particular forms of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. A composition intended to deliver the particle to follicles in the dermis, the composition comprising host particles and metal particles, wherein the composition exhibits a greater than 70% attachment of the metal particles to the host particles.

2. The composition of claim 1, wherein the pH of the composition is between 6 and 8.

3. The composition of claim 1, wherein the composition further comprises of a singular or mixture of preservatives.

4. The composition of claim 3, wherein the preservative is selected from the group consisting of LAE, Benzyl Alcohol, MIT, Methyl, Ethyl, propyl parabens, Dehydroacetic Acid and 1,2-Hexane diol, phenoxyethanol and combinations thereof.

5. The composition of claim 4, wherein the composition further comprises between 0.01% LAE and 1% LAE.

6. The composition of claim 4, wherein the composition comprises between 0.3% Benzyl Alcohol and 1.0% Benzyl Alcohol.

7. The composition of claim 1, wherein the host particles are silica particles, wherein the silica particles are between 300 nm to 1000 nm in diameter.

8. The composition of claim 1 wherein the ratio of host particle to metal nanoparticle o between and inclusive of 1.5:1 to 6.5:1.

9. The composition of claim 1, wherein the metal particles comprise gold nanorods, silver nanorods, or combinations thereof.

10. The composition of claim 1, wherein the metal particles are one or more gold nanorods, each of the gold nanorods having a peak max absorption of between 700 nm to 1100 nm.

11. A composition intended to deliver the particle to follicles in the dermis, the composition comprising silica particles and gold nanorod particles at an attachment level of greater than 70% of the gold nanorod particles to the silica particles.

12. The composition of claim 11, wherein the pH of the composition is between 6 and 8.

13. The composition of claim 11, wherein the composition further comprises of a singular or mixture of preservatives.

14. The composition of claim 13, wherein the preservative is selected from the group consisting of LAE, Benzyl Alcohol, MIT, Methyl Paraben, Ethyl Paraben, Propyl paraben, Dehydroacetic Acid and 1,2-Hexane diol, phenoxyethanol and combinations thereof.

15. The composition of claim 14, wherein the composition further comprises between 0.01% LAE and 0.5% LAE.

16. The composition of claim 14, wherein the composition comprises between 0.3% Benzyl Alcohol and 1% Benzyl Alcohol.

17. The composition of claim 11, wherein the silica particles are between 300 nm to 1000 nm in diameter.

18. The composition of claim 11, wherein the ratio of host particle to gold nanorod is between and inclusive of 1.5:1 to 6.5:1.

19. The composition of claim 11, wherein the gold nanorods have a peak max absorption between 700 nm to 1100 nm.

20. The composition of claim 11, wherein the composition is in the form of a suspension, a gel, milk, a cream, an ointment, a lotion, a foam, a soft paste, or any other cosmetically acceptable form.
